(19) United States
(12) Patent Application Publication
(10) Pub. No.: US 2025/0188175 A1
(43) Pub. Date: Jun. 12, 2025

(54) LOW-DOSE TOCILIZUMAB FOR THE TREATMENT OR PREVENTION OF SICKLE CELL CRISIS

(71) Applicant: The University of Chicago, Chicago, IL (US)

(72) Inventor: Mark J. RATAIN, Chicago, IL (US)

(21) Appl. No.: 18/971,607

(22) Filed: Dec. 6, 2024

Related U.S. Application Data
(60) Provisional application No. 63/606,970, filed on Dec. 6, 2023.

(51) Int. Cl. C07K 16/28; A61K 39/00

(52) U.S. Cl. CPC C07K 16/2866; A61K 2039/545; C07K 2317/24

(57) ABSTRACT

Provided herein are methods for the treatment or prevention of sickle cell crisis and symptoms associated therewith by the administration of low doses of tocilizumab to a subject. In particular embodiments, provided herein are pharmaceutical compositions comprising doses of 200 mg or less of tocilizumab and methods for the treatment or prevention of sickle cell crisis and symptoms or other conditions arising as a result thereof.

### CROSS REFERENCE TO RELATED APPLICATION

The present application claims benefit to U.S. Provisional Patent Application No. 63/606,970, filed on Dec. 6, 2023, of which is incorporated by reference in its entirety.

### FIELD

Provided herein are methods for the treatment or prevention of sickle cell crisis and symptoms associated therewith by the administration of low doses of tocilizumab to a subject. In particular embodiments, provided herein are pharmaceutical compositions comprising doses of 200 mg or less of tocilizumab and methods for the treatment or prevention of sickle cell crisis and symptoms or other conditions arising as a result thereof.

### BACKGROUND

Sickle cell disease (SCD) is a group of, genetically inherited, blood disorders, resulting in an abnormality in the oxygen-carrying protein hemoglobin found in red blood cells, leading to a rigid, sickle-like shape under certain circumstances. Symptoms of sickle cell disease typically begin around 5 to 6 months of age, and a number of health problems may develop, such as anemia, swelling in the hands and feet, bacterial infections, stroke, and attacks of pain, known as a sickle cell crisis.

Sickle cell crisis is the primary clinical feature of sickle cell disease and often requires hospitalization. In general, a sickle cell crisis occurs when sickle-shaped red blood cells clump together and block small blood vessels that carry blood to certain organs, muscles, and bones. This causes mild to severe pain. The pain can last from hours to days. The term “sickle cell crisis” is used to describe several acute conditions such as the vaso-occlusive crisis (acute painful crisis), aplastic crisis, splenic sequestration crisis, hyperhemolytic crisis, hepatic crisis, dactylitis, priapism and acute chest syndrome.

### SUMMARY

Provided herein are methods for the treatment or prevention of sickle cell crisis and symptoms associated therewith by the administration of low doses of tocilizumab to a subject. In particular embodiments, provided herein are pharmaceutical compositions comprising doses of 200 mg or less of tocilizumab and methods for the treatment or prevention of sickle cell crisis and symptoms or other conditions arising as a result thereof.

In some embodiments, provided herein are methods of treating a subject suffering from sickle cell crisis comprising administering an effective dose of 200 mg or less of tocilizumab to the subject. In some embodiments, the subject suffers from vaso-occlusive crisis, aplastic crisis, splenic sequestration crisis, hemolytic crisis, hyperhemolytic crisis, hepatic crisis, dactylitis, priapism, and/or acute chest syndrome. In some embodiments, the subject is an adult patient. In some embodiments, the subject is a pediatric patient.

In some embodiments, an effective dose comprises 200 mg or less of tocilizumab (e.g., 10 mg, 20 mg, 30, mg, 40 mg, 50 mg, 60 mg, 70 mg, 80 mg, 90 mg, 100 mg, 110 mg, 120 mg, 130, mg, 140 mg, 150 mg, 160 mg, 170 mg, 180 mg, 190 mg, 200 mg, or ranges therebetween). In some embodiments, an effective dose comprises less than 100 mg of tocilizumab. In some embodiments, an effective dose comprises less than 80 mg of tocilizumab. In some embodiments, an effective dose comprises less than 40 mg of tocilizumab. In some embodiments, an effective dose comprises less than 20 mg of tocilizumab. In some embodiments, tocilizumab is administered daily. In some embodiments, tocilizumab is administered two or more times daily (e.g., 2, 3, 4, or more times). In some embodiments, tocilizumab is administered on alternate days or every third or fourth day. In some embodiments, tocilizumab is administered subcutaneously, intravenously, or by any other suitable method.

In some embodiments, a subject is administered multiple doses (e.g., over the course of 2 days, 3 days, 4 days, 5 days, 6 days, 1 week, 2 weeks, or more, or ranges therebetween). In some embodiments, each dose administered is 200 mg or less of tocilizumab (e.g., 10 mg, 20 mg, 30, mg, 40 mg, 50 mg, 60 mg, 70 mg, 80 mg, 90 mg, 100 mg, 110 mg, 120 mg, 130, mg, 140 mg, 150 mg, 160 mg, 170 mg, 180 mg, 190 mg, 200 mg, or ranges therebetween).

In some embodiments, the subject is administered multiple doses comprising 200 mg or less total of tocilizumab (e.g., 200 mg, or ranges therebetween). In some embodiments, the multiple doses total less than 200 mg of tocilizumab (e.g., 175 mg, 150 mg, 125 mg, 100 mg, or ranges therebetween).

In some embodiments, tocilizumab is co-administered with one or more additional therapeutics.

In some embodiments, methods comprise: (a) testing a subject or a biological sample from a subject to determine the appropriateness of tocilizumab administration to treat/prevent sickle cell crisis; and (b) administering a dose of tocilizumab consistent with the methods herein. In some embodiments, testing comprises determining the subject's C-reactive protein level. In some embodiments, testing comprises determining the subject's serum interleukin-6 (IL-6) level. In some embodiments, methods further comprise: (c) testing a subject or a biological sample from a subject to determine the effectiveness of the tocilizumab administration. In some embodiments, methods further comprise: (d) determining a treatment course of action based on the testing of step (a) and/or step (c).

In some embodiments, provided herein is the use of an effective dose of 200 mg or less of tocilizumab for treating a subject suffering from sickle cell crisis.

In some embodiments, provided herein is the use of an effective dose of 200 mg or less of tocilizumab in the manufacture of a medicament for use in a method of treating a subject suffering from sickle cell crisis.

In some embodiments, provided herein are methods of preventing or reducing the likelihood of sickle cell crisis in a subject comprising administering a recurring effective dose of 200 mg or less of tocilizumab to the subject. In some embodiments, the effective dose is administered once every 1-8 weeks. In some embodiments, the effective dose is administered about every 4 weeks. In some embodiments, the recurring effective dose comprises less than 100 mg of tocilizumab. In some embodiments, the recurring effective dose comprises less than 50 mg of tocilizumab. In some embodiments, methods herein further comprise testing the subject or a biological sample from a subject to determine the appropriateness of a preventative tocilizumab to prevent sickle cell crisis. In some embodiments, testing comprises determining the subject's C-reactive protein level. In some embodiments, testing comprises determining the subject's serum and/or sputum IL-6 level.

### Definitions

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “subject” broadly refers to any animal, including but not limited to, human and non-human animals (e.g., dogs, cats, cows, horses, sheep, poultry, fish, crustaceans, etc.). As used herein, the term “patient” typically refers to a subject that is being treated for a disease or condition. In most embodiments herein, the subject is a human subject.

As used herein, the terms “administration” and “administering” refer to the act of giving a drug, prodrug, or other agent, or therapeutic treatment to a subject or in vivo, in vitro, or ex vivo cells, tissues, and organs. Exemplary routes of administration to the human body can be by parenteral administration (e.g., intravenously, subcutaneously, etc.).

As used herein, the term “effective amount” refers to the amount of a composition sufficient to effect beneficial or desired results. An effective amount can be administered in one or more administrations, applications or dosages and is not intended to be limited to a particular formulation or administration route.

As used herein, the terms “co-administration” and “co-administering” refer to the administration of at least two agent(s) (e.g., tocilizumab and one or more additional therapeutics) or therapies to a subject. In some embodiments, the co-administration of two or more agents or therapies is concurrent (e.g., in a single formulation/composition or in separate formulations/compositions). In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

The terms “prevent,” “prevention,” “prophylactic,” and the like refer to completely or partially inhibiting onset of a disease or symptom thereof (e.g., sickle cell crisis). A “prophylactically effective amount” of a particular compound, drug, or agent refers to an amount effective, at dosages and for periods of time necessary, to achieve a desired prophylactic result (e.g., prevention of disease onset). The likelihood of developing a condition need not be completely eliminated to constitute prevention. If a composition or method step reduces the likelihood of developing a condition across a population, then the composition or method step prevents the condition within the scope herein (e.g., when administered to an individual).

As used herein, the terms “treatment,” “treating,” and the like refer to obtaining a desired pharmacologic and/or physiologic effect. Preferably, the effect is therapeutic, i.e., the effect partially or completely cures a disease and/or adverse symptom attributable to the disease (e.g., sickle cell crisis). A “therapeutically effective amount” of a particular compound, drug, or agent refers to an amount effective, at dosages and for periods of time necessary, to achieve a desired therapeutic result. A therapeutically effective amount may vary according to factors such as the disease state, age, sex, and weight of a subject.

As used herein, the term “pharmaceutical composition” refers to the combination of an active agent with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “instructions for administering,” and grammatical equivalents thereof, includes instructions for using the compositions contained in a kit for the treatment of conditions (e.g., providing dosing, route of administration, decision trees for treating physicians for correlating patient-specific characteristics with therapeutic courses of action).

As used herein, the term “antibody” refers to a whole antibody molecule or a fragment thereof (e.g., fragments such as Fab, Fab′, and F(ab′)₂), it may be a polyclonal or monoclonal antibody, a chimeric antibody, a humanized antibody, a human antibody, etc.

As used herein, the terms “anti-IL-6 receptor antibody” or “IL-6 receptor antibody” refer to an antibody which specifically recognizes an antigen and/or epitope presented by the IL-6 receptor.

As used herein, the term “monoclonal antibody” refers to an antibody which is a member of a substantially homogeneous population of antibodies that specifically bind to the same epitope. In certain embodiments, a monoclonal antibody is secreted by a hybridoma. In certain such embodiments, a hybridoma is produced according to certain methods known to those skilled in the art. See, e.g., Kohler and Milstein (1975) Nature 256:495-499; herein incorporated by reference in its entirety. In certain embodiments, a monoclonal antibody is produced using recombinant DNA methods (see, e.g., U.S. Pat. No. 4,816,567). In certain embodiments, a monoclonal antibody refers to an antibody fragment isolated from a phage display library. See, e.g., Clackson et al. (1991) Nature 352:624-628; and Marks et al. (1991) J. Mol. Biol. 222:581-597; herein incorporated by reference in their entireties. The modifying word “monoclonal” indicates properties of antibodies obtained from a substantially-homogeneous population of antibodies, and does not limit a method of producing antibodies to a specific method. For various other monoclonal antibody production techniques, see, e.g., Harlow and Lane (1988) Antibodies: A Laboratory Manual (Cold Spring Harbor Laboratory, Cold Spring Harbor, N.Y.); herein incorporated by reference in its entirety.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Suitable samples that may find use in embodiments herein include, but are not limited to: blood, plasma, sera, urine, saliva, cells, cell lysates, tissues, tissue homogenates, cerebrospinal fluid, etc.

As used herein, the term “biosimilar” refers to a biopharmaceutical which is deemed to be comparable in quality, safety, and efficacy to a reference product marketed by an innovator company (Section 351 (i) of the Public Health Service Act (42 U.S.C. 262 (i)). Tocilizumab biosimilars may find use in any suitable embodiments herein.

### DETAILED DESCRIPTION

Provided herein are methods for the treatment or prevention of sickle cell crisis and symptoms associated therewith by the administration of low doses of tocilizumab to a subject. In particular embodiments, provided herein are pharmaceutical compositions comprising doses of 200 mg or less of tocilizumab and methods for the treatment or prevention of sickle cell crisis and symptoms or other conditions arising as a result thereof.

Tocilizumab is a monoclonal antibody that competitively inhibits the binding of IL-6 to its receptor (IL-6R). Inhibiting the entire receptor complex prevents IL-6 signal transduction to inflammatory mediators that summon B and T cells. Tocilizumab was originally developed for outpatient, every four-weeks administration in rheumatoid arthritis (RA), giant cell arteritis, polyarticular juvenile idiopathic arthritis, and systemic juvenile idiopathic arthritis at a labeled dose of 8 mg/kg, with approval based on improvement in the American College of Rheumatology (ACR) 20 score.

A recent study has identified that IL-6 levels are dramatically high in the sputum from children with sickle cell disease during acute chest syndrome (Allali et al. Blood Adv. 2020 Dec. 22; 4 (24): 6130-6134; incorporated by reference in its entirety).

Provided herein are compositions (e.g., comprising tocilizumab) for the treatment of sickle cell crisis and symptoms/conditions arising therefrom comprising unit doses of tocilizumab in the range of 10-200 mg, with a maximum total combined dose of 200 mg per month. In some embodiments, provided herein are methods (e.g., administering tocilizumab or co-administering tocilizumab with additional therapies) for the treatment of sickle cell crisis and symptoms/conditions arising therefrom comprising administering unit doses of tocilizumab in the range of 10-200 mg, repeated at intervals (e.g., 24 hours, 48 hours, etc.) as required for up to ten doses (e.g., 2, 3, 4, 5, 6, 7, 8, 9, 10, or ranges therebetween), with a maximum total dose of, for example, 200 mg. In some embodiments, tocilizumab is administered either intravenously or subcutaneously. In some embodiments, dosing is guided by laboratory and/or clinical studies (e.g., CRP and IL-6 assays).

Sickle cell crisis, as used herein, may include vaso-occlusive crisis, aplastic crisis, splenic sequestration crisis, hemolytic crisis, and others.

Vaso-occlusive crisis is caused by sickle-shaped red blood cells that obstruct capillaries and restrict blood flow to an organ, resulting in ischemia, pain, necrosis, and often organ damage. The frequency, severity, and duration of these crises vary considerably. Painful crises are treated with hydration, analgesics, and blood transfusion; pain management requires opioid drug administration at regular intervals until the crisis has settled. For milder crises, a subgroup of patients typically manages on nonsteroidal anti-inflammatory drugs such as diclofenac or naproxen. For more severe crises, most patients require inpatient management for intravenous opioids; patient-controlled analgesia devices are commonly used in this setting. Vaso-occlusive crisis involving organs such as the penis (i.e., priapism) or lungs are considered an emergency and treated with red blood cell transfusions. Incentive spirometry, a technique to encourage deep breathing to minimize the development of atelectasis, is recommended in some cases. In some embodiments, a low-dose treatment with tocilizumab, as described herein, is administered for the treatment of vaso-occlusive crisis. In some embodiments, a low-dose treatment with tocilizumab, as described herein, is co-administered with any of the above therapies for the treatment of vaso-occlusive crisis.

The spleen is frequently affected in sickle cell disease, as the sickle-shaped red blood cells causes narrowing of blood vessels and reduced function in clearing the defective cells. It is usually infarcted before the end of childhood in individuals suffering from sickle cell anemia. This spleen damage increases the risk of infection from encapsulated organisms; preventive antibiotics and vaccinations are recommended for those lacking proper spleen function. Splenic sequestration crises are acute, painful enlargements of the spleen, caused by intrasplenic trapping of red cells and resulting in a precipitous fall in hemoglobin levels with the potential for hypovolemic shock. Sequestration crises are considered an emergency. If not treated, patients may die within 1-2 hours due to circulatory failure. Management is supportive, sometimes with blood transfusion. These crises are transient; they continue for 3-4 hours and may last for one day. In some embodiments, a low-dose treatment with tocilizumab, as described herein, is administered for the treatment of splenic crisis. In some embodiments, a low-dose treatment with tocilizumab, as described herein, is co-administered with any of the above therapies for the treatment of splenic crisis.

Acute chest syndrome is defined by at least two of these signs or symptoms: chest pain, fever, pulmonary infiltrate or focal abnormality, respiratory symptoms, or hypoxemia. It is the second-most common complication and it accounts for about 25% of deaths in patients with SCD. Most cases present with vaso-occlusive crises, and then develop acute chest syndrome. Nevertheless, about 80% of people have vaso-occlusive crises during acute chest syndrome. In some embodiments, a low-dose treatment with tocilizumab finds use in the treatment of splenic crisis.

Aplastic crises are acute worsenings of the patient's baseline anemia, producing pale appearance, fast heart rate, and fatigue. This crisis is normally triggered by parvovirus B19, which directly affects production of red blood cells by invading the red cell precursors and multiplying in and destroying them. Parvovirus infection almost completely prevents red blood cell production for two to three days. In normal individuals, this is of little consequence, but the shortened red cell life of SCD patients results in an abrupt, life-threatening situation. Reticulocyte counts drop dramatically during the disease (causing reticulocytopenia), and the rapid turnover of red cells leads to the drop in hemoglobin. This crisis takes 4 to 7 days to disappear. Most patients can be managed supportively; some need a blood transfusion. In some embodiments, a low-dose treatment with tocilizumab finds use in the treatment of aplastic crises.

Hemolytic crises are acute accelerated drops in hemoglobin level. The red blood cells break down at a faster rate. This is particularly common in people with coexistent glucose-6-phosphate dehydrogenase (G6PD) deficiency, or in patients who have recently received transfusions of packed red blood cells. Management is supportive, sometimes with blood transfusions. In some embodiments, a low-dose treatment with tocilizumab finds use in the treatment of hemolytic crises.

Sickle cell crisis patient evaluation warrants routine laboratory examination such as complete blood count (CBC) with differential, a reticulocyte count, and a complete metabolic panel including liver function tests. Type and screen blood for possible transfusion if needed. Inflammatory markers include CRP, procalcitonin, and ferritin, and cultures of blood and other fluids may be considered for fever and identification of the source of infection.

Tocilizumab is typically administered (e.g., for treatment of adults with moderate-to-severe active rheumatoid arthritis (RA)) via intravenous (IV) infusion of subcutaneous injection. For IV infusion, a standard dose for the treatment of RA is 4 mg/kg (e.g., over 60 min) every four weeks (q4Weeks), which may be increased to 8 mg/kg q4Weeks based on clinical response. The IV dose of tocilizumab for the treatment of RA is not to exceed 800 mg/dose every four weeks. Typical doses of tocilizumab for the treatment of RA are 400-800 mg every four weeks. At an individual patient level, utilizing the minimally effective dose of a drug (tocilizumab) can optimize efficacy with minimalization of adverse events. In some embodiments, provided herein are lower than standard RA doses of tocilizumab for the treatment or prevention of sickle cell crisis and symptoms/conditions arising therefrom.

In some embodiments, tocilizumab is provided in 10-200 mg doses (e.g., 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, and ranges therebetween (e.g., 20-80 mg)). In some embodiments, the dose is adjusted based on the effectiveness and/or side effects of initial dosing. For example, if the initial dose is/appears effective and/or side effects are/appear significant, the dose may be reduced (e.g., 10-100 mg (e.g., 10 mg, 20 mg, 30 mg, 40 mg, 50 mg, 60 mg, 70 mg, 80 mg, 90 mg, 100 mg, and ranges therebetween (e.g., 30-50 mg)). If the initial dose is/appears ineffective and/or side effects are/appear insignificant, the dose may be increased (e.g., 100-200 mg (e.g., 100 mg, 110 mg, 120 mg, 130 mg, 140 mg, 150 mg, 160 mg, 170 mg, 180 mg, 190 mg, 200 mg, and ranges therebetween).

In some embodiments, dosing is guided by clinical and/or laboratory studies, with a particular focus on CRP and IL-6 assays.

In some embodiments, the dose is adjusted based on the effectiveness and/or side effects of initial dosing. For example, if the initial dose is/appears effective and/or side effects are/appear significant, the dose may be reduced (e.g., 20-100 mg (e.g., 20 mg, 30 mg, 40 mg, 50 mg, 60 mg, 70 mg, 80 mg, 90 mg, 100 mg, and ranges therebetween (e.g., 30-50 mg)). If the initial dose is/appears ineffective and/or side effects are/appear insignificant, the dose may be increased.

In some embodiments, the aforementioned doses are administered daily, or once every 2 days, or 3 days. In some embodiments, doses are administered based on symptoms, biomarkers, and/or clinical evaluation.

In some embodiments, 200 mg or less of tocilizumab is administered daily (q1 day) (e.g., 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, or ranges therebetween).

In some embodiments, 200 mg or less of tocilizumab is administered on alternating days (q2 day) (e.g., 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, or ranges therebetween).

In some embodiments, doses are administered q1 day, q2 day, q3 day, q4 day, q5 day, q6 day, q1 week, q2 week, q3 week, q4 week, q5 week, q6 week, or any regimens therebetween.

In some embodiments, a first dose (e.g., of less than 200 mg (e.g., 50 mg, 80 mg, 200 mg, ranges therebetween, etc.) of tocilizumab is administered. In some embodiments, a second dose (e.g., of less than 200 mg (e.g., 50 mg, 80 mg, 150 mg, ranges therebetween, etc.) of tocilizumab is administered 24 hours (e.g., plus or minus 1, 2, 4, 6, or 8 hours) after the first dose. In some embodiments, a second dose (e.g., of less than 200 mg (e.g., 50 mg, 80 mg, 150 mg, ranges therebetween, etc.) of tocilizumab is administered 36 hours (e.g., plus or minus 1, 2, 4, 6, or 8 hours) after the first dose. In some embodiments, a second dose (e.g., of less than 200 mg (e.g., 50 mg, 80 mg, 150 mg, ranges therebetween, etc.) of tocilizumab is administered 48 hours (e.g., plus or minus 1, 2, 4, 6, or 8 hours) after the first dose. In some embodiments, a second dose (e.g., of less than 200 mg (e.g., 50 mg, 80 mg, 150 mg, ranges therebetween, etc.) of tocilizumab is administered 72 hours (e.g., plus or minus 1, 2, 4, 6, or 8 hours) after the first dose. In some embodiments, a subsequent dose (e.g., after a first, second, third, etc.) is administered with a 4 hours, 8 hours, 12 hours, 24 hours, 2 days, 3 days, 4 days, 5 days, 6 days, 1 week, 2 weeks, 3 weeks, 4 weeks, etc. between administrations.

In some embodiments, methods are provided for the treatment or prevention of sickle cell crisis by the co-administration of tocilizumab with one or more additional therapeutics or therapies. In some embodiments, tocilizumab is co-administered with hydroxyurea (Droxia, Hydrea, Siklos), L-glutamine oral powder (Endari), crizanlizumab (Adakveo), pain-relieving medications (e.g., analgesics, NSAIDS, opiods, etc.), Voxelotor (Oxbryta), or other therapeutics or therapies discussed herein.

In some embodiments, methods are provided for testing, assessing, quantifying, qualifying, evaluating, etc. one or more signs, symptoms, biomarkers, etc. of sickle cell crisis. Any techniques described herein (e.g., chest x-ray, qPCR, ELISA, etc.) for the detection of biomarkers, symptoms, etc. are within the scope herein.

In some embodiments, methods herein comprise administering a dose of tocilizumab and subsequently testing a subject for the presence of one or more symptoms and/or biomarkers of sickle cell crisis to determine the success of the treatment.

In some embodiments, provided herein are multiple rounds of testing and treating (e.g., test-treat-test, treat-test-treat, etc.).

Some embodiments herein comprise a step of identifying a subject as suffering from sickle cell crisis using a suitable assay or kit.

In some embodiments, C-reactive protein (CRP) is measured as a useful proxy marker for serum IL-6 levels.

### REFERENCES

The following references are herein incorporated by reference in their entireties.
Strohbehn et al. Applied Clinical Pharmacology in a Crisis: Interleukin-6 Axis Blockade and COVID-19. Clin Pharmacol Ther. 2020 September; 108 (3): 425-427.
Hanan Kifah Abdul-Hussein et al. Evaluation of the expression of red blood cell CD36, interleukin-6 and interleukin-8 in sickle cell anemia pediatric patients. Cytokine 143 (2021) 155534.
Domingos et al. High levels of proinflammatory cytokines IL-6 and IL-8 are associated with a poor clinical outcome in sickle cell anemia. Annals of Hematology (2020) 99:947-953.
Naprawa et al. Serum Biomarkers for Identifying Acute Chest Syndrome Among Patients Who Have Sickle Cell Disease and Present to the Emergency Department. Pediatrics. 2005 September; 116 (3): e420-5.
Elzubeir et al. Estimation of Serum Interleukin-6 level as a Useful Marker for Clinical Severity of Sickle Cell Disease among Sudanese Patients. Int. J. of Multidisciplinary and Current research, Vol. 5 (May/June 2017).
Niu et al. Angiogenic and Inflammatory Markers of Cardiopulmonary Changes in Children and Adolescents with Sickle Cell Disease. PLOS One. 2009 Nov. 23; 4 (11): e7956.
Odievre et al. Dramatic improvement after tocilizumab of severe COVID-19 in a child with sickle cell disease and acute chest syndrome. Am J Hematol. 2020 August; 95 (8): E192-E194.
De Luna et al. Rapid and severe Covid-19 pneumonia with severe acute chest syndrome in a sickle cell patient successfully treated with tocilizumab. Am J Hematol. 2020; 1-3.
Allali et al. IL-6 levels are dramatically high in the sputum from children with sickle cell disease during acute chest syndromeBlood Adv. 2020 Dec. 22; 4 (24): 6130-6134.
Strohbehn et al. COVIDOSE: A Phase II Clinical Trial of Low-Dose Tocilizumab in the Treatment of Noncritical COVID-19 Pneumonia. Clin Pharmacol Ther. 2021 March; 109 (3): 688-696.

1. A method of treating a subject suffering from sickle cell crisis comprising administering an effective dose of 200 mg or less of tocilizumab to the subject.

2. The method of claim 1, wherein the subject suffers from vaso-occlusive crisis, aplastic crisis, splenic sequestration crisis, hemolytic crisis, hyperhemolytic crisis, hepatic crisis, dactylitis, priapism, and/or acute chest syndrome.

3. The method of claim 1, wherein the effective dose comprises less than 100 mg of tocilizumab.

4. The method of claim 1 wherein the effective dose comprises less than 50 mg of tocilizumab.

5. The method of claim 1, wherein the subject is administered multiple doses over one month or less comprising less than 200 mg total of tocilizumab.

6. The method of claim 5, wherein the multiple doses total less than 100 mg of tocilizumab.

7. The method of one of claims 1-6, wherein tocilizumab is administered once daily.

8. The method of one of claims 1-6, wherein tocilizumab is administered once every 2-3 days.

9. The method of one of claims 1-8, wherein tocilizumab is administered subcutaneously.

10. The method of one of claims 1-8, wherein tocilizumab is administered intravenously.

11. The method of one of claims 1-25, wherein the tocilizumab is co-administered with one or more additional therapeutics.

12. A method comprising:
(a) testing a subject or a biological sample from a subject to determine the appropriateness of tocilizumab administration to treat/prevent sickle cell crisis;
(b) administering a dose of tocilizumab consistent with the methods of one of claims 1-11.

13. The method of claim 12, wherein testing comprises determining the subject's C-reactive protein level.

14. The method of claim 12, wherein testing comprises determining the subject's serum and/or sputum IL-6 level.

15. The method of claim 12, further comprising:
(c) testing a subject or a biological sample from a subject to determine the effectiveness of the tocilizumab administration.

16. The method of claim 15, further comprising:
(d) determining a treatment course of action based on the testing of step (a) and/or step (c).

17. Use of an effective dose of 200 mg or less of tocilizumab for treating a subject suffering from sickle cell crisis.

18. Use of an effective dose of 200 mg or less of tocilizumab in the manufacture of a medicament for use in a method of treating a subject suffering from sickle cell crisis.

19. A method of preventing or reducing the likelihood of sickle cell crisis in a subject comprising administering a recurring effective dose of 200 mg or less per month of tocilizumab to the subject.

20. The method of claim 19, wherein the effective dose is administered once every 1-8 weeks.

21. The method of claim 20, wherein the effective dose is administered about every 4 weeks.

22. The method of claim 20, wherein the recurring effective dose comprises less than 100 mg of tocilizumab.

23. The method of claim 20 wherein the recurring effective dose comprises less than 50 mg of tocilizumab.

24. The method of claim 20, further comprising testing the subject or a biological sample from a subject to determine the appropriateness of tocilizumab administration to prevent sickle cell crisis.

25. The method of claim 24, wherein testing comprises determining the subject's C-reactive protein level.

26. The method of claim 24, wherein testing comprises determining the subject's serum and/or sputum IL-6 level.
